(19) United States
(12) Patent Application Publication
(10) Pub. No.: US 2015/0087606 A1
(43) Pub. Date: Mar. 26, 2015

(54) PHARMACEUTICAL COMPOSITION FOR PREVENTING OR TREATING AMYLOID BETA PEPTIDE-ASSOCIATED DISEASES OR CONDITIONS

(71) Applicant: Sinphar Tian-Li Pharmaceutical Co., Ltd. (Hangzhou), Hangzhou (CN)

(72) Inventors: Hang-Ching Lin, Taipei City (TW); Muh-Hwan Su, I Lan (TW); Young-Ming Huang, I Lan (TW); Jing-Jing Tang, I Lan (TW)

(21) Appl. No.: 14/365,404

(22) PCT Filed: Dec. 17, 2012
(86) PCT No.: PCT/CN2012/086796

§ 371 Date: Jun. 13, 2014

Related U.S. Application Data
(60) Provisional application No. 61/576,367, filed on Dec. 16, 2011.

(51) Int. Cl. A61K 31/7032; A23G 4/10; A23L 2/52; A23L 1/30

(52) U.S. Cl. CPC A61K 31/7032; A23L 1/3002; A23G 4/10; A23L 2/52
USPC 514/25

(57) ABSTRACT

A pharmaceutical composition containing isoacteoside to the acteoside is provided, which is able to inhibit formation, accumulation or aggregation of amyloid β peptides, and is thus useful in preventing or treating amyloid beta peptide-associated diseases or conditions, wherein a weight ratio of the isoacteoside to the acteoside is 4:1 to 1:4.

### CROSS REFERENCE TO RELATED APPLICATIONS

This application is the National Stage of International Application No. PCT/CN2012/086796, filed on Dec. 17, 2012, which claims the benefit of U.S. Provisional Application No. 61/576,367, filed on Dec. 16, 2011. The contents of both applications are hereby incorporated by reference in their entirety.

### TECHNICAL FIELD

The present invention relates to a pharmaceutical composition for use in preventing or treating amyloid β peptide associated diseases or conditions, which comprises acteoside and isoacteoside as potent components capable of inhibiting formation, accumulation or aggregation of amyloid beta peptides.

### BACKGROUND TECHNIQUES

U.S. Pat. No. 7,087,252 B2 discloses a medicinal preparation containing phenylethanoid glycosides extracted from Cistanche tubulosa (Schenk.) Wight, said preparation comprising 25-50 wt % of echinacoside and 5-15 wt % of acteoside, which is useful in treating senile dementia. Isoacteoside and other phenylethanoid glycosides are known also being contained in said medicinal preparation.

The applicant of this application in WO 2011/157059 A1 discloses use of isoacteoside or a pharmaceutically acceptable salt thereof in inhibiting the formation, accumulation or aggregation of amyloid β peptide (Aβ), and use in the fabrication of a medicament for preventing or treating Aβ-associated diseases or conditions.

The full disclosures in U.S. Pat. No. 7,087,252 B2 and WO 2011/157059 A1 are incorporated herein by reference.

In the present application, the inventors continue the research of WO 2011/157059 A1 and obtain a related inventive accomplishment.

### SUMMARY OF THE INVENTION

Since Aβ and its aggregates are likely to cause various diseases or conditions in organisms, one object of the present invention is to provide pharmaceutical composition for inhibiting formation, accumulation or aggregation of Aβ, and such pharmaceutical composition can be used as an additive in food, drinks, chewing substance, patches, skin care products, etc. Another object of the present invention is to provide a pharmaceutical composition for preventing or treating Aβ-associated diseases or conditions.

Still another object of the present invention is to provide use of a pharmaceutical composition in the fabrication of a medicament for preventing or treating Aβ-associated diseases or conditions.

A pharmaceutical composition for preventing or treating Aβ-associated diseases or conditions provided in accordance with the present invention comprises acteoside and isoacteoside as potent components, wherein a weight ratio of the isoacteoside to the acteoside is 4:1 to 1:4.

Preferably, the weight ratio of the isoacteoside to the acteoside in the pharmaceutical composition is 4:1 to 2:3.

Preferably, the pharmaceutical composition is free of echinacoside.

Preferably, the pharmaceutical composition is able to inhibit formation, accumulation or aggregation of amyloid β peptides.

Preferably, the pharmaceutical composition is able to inhibit extracellular formation, accumulation or aggregation of amyloid β peptides.

Preferably, the pharmaceutical composition is able to inhibit neuronal damage or apoptosis caused by the amyloid β peptides, so as to retain, improve or restore learning and memory abilities.

Preferably, the Aβ-associated disease or condition is Alzheimer's disease, mild cognitive impairment, Lewy body dementia, Down syndrome, Hereditary cerebral hemorrhage with amyloid (HCHWA) Dutch, Parkinsonism-dementia complex on Guam, Cerebral amyloid angiopathy, inclusion body myositis, frontotemporal dementia, age-related macular degeneration, or Pick's disease.

Preferably, the pharmaceutical composition is for treating Alzheimer's disease.

cPreferably, the pharmaceutical composition is for preventing an organism from suffering Alzheimer's disease or for delaying an organism suffering Alzheimer's disease.

Preferably, an effective dosage of the pharmaceutical composition to a person is equivalent to per day 0.2 mg to 4.0 mg of the potent components per kg of body weight.

Preferably, the pharmaceutical composition comprises a phenylethanoid glycoside preparation extracting from a plant as a source of the potent components, wherein the preparation comprises the isoacteoside to the acteoside as the major phenylethanoid glycosides, and the content of the isoacteoside is greater than that of the acteoside.

Preferably, the preparation comprises 12-32% of acteoside and 26-46% of the isoacteoside, based on the weight of the preparation.

Preferably, the plant is Cistanche tubulosa (Schenk.) Wight.

Preferably, the preparation is provided by a process comprising the following steps:

a) extracting fleshy stems of Cistanche tubulosa (Schenk.) Wight with a first polar solvent;

b) introducing the resulting extract from step a) into a column which is packed with hydrophobic macro-porous polymeric beads, thereby enabling phenylethanoid glycosides to be adsorbed on the polymeric beads;

c) eluting the column by use of a second polar solvent serving as a mobile phase, so that relatively less strongly adsorbed compounds are eluted from the column with most of phenylethanoid glycosides still being adsorbed on the polymeric beads; and

d) eluting the column by use of a third polar solvent so as to obtain an eluate which contains phenylethanoid glycosides, wherein the first polar solvent is water, methanol, ethanol, a mixture of water and methanol, or a mixture of water and ethanol; the second polar solvent is water; and the third polar solvent is methanol, ethanol, a mixture of water and methanol, or a mixture of water and ethanol, and the third polar solvent is lower in polarity than the second polar solvent;

e) concentrating the eluate which contains phenylethanoid glycosides, dissolving the concentrate in water, and contacting the aqueous solution with a macro-porous resin, so that the phenylethanoid glycosides are adsorbed on the macro-porous resin; and

f) eluting the macro-porous resin with a fourth polar solvent and a fifth polar solvent in sequence, wherein the fifth polar solvent is lower in polarity than the fourth polar solvent, so that an eluate resulting from the fourth polar solvent elution does not contain acteoside and isoacteoside, and an eluate resulting from the fifth polar solvent elution contain only acteoside and isoacteoside, wherein the fourth polar solvent and the fifth polar solvent are a mixture of water and methanol or a mixture of water and ethanol.

Preferably, the fourth polar solvent is 25-35% ethanol aqueous solution and the fifth polar solvent is 35-45% ethanol aqueous solution.

To better understand the above and other objects, features and advantages of the present invention, the present invention will be described in detail below with examples presented with reference to the annexed drawings.

### BRIEF DESCRIPTION OF THE DRAWINGS

FIG. 1 shows the effects of drug A (acteoside), drug I (isoacteoside), C (control group, no drug), and pharmaceutical compositions having different ratios of A to I on extracellular Aβ1-40 accumulation.

FIG. 2 shows the effects of drug A (acteoside), drug I (isoacteoside), C (control group, no drug), and pharmaceutical compositions having different ratios of A to I on Aβ1-42 on Aβ1-42 oligomerization.

### BEST MODES OF EMBODYING THE INVENTION

Various diseases caused by Aβ have a common feature: formation of Aβ aggregates. These Aβ aggregates present in shapes such as fibrils or plaques, and deposit in systems, organs, tissues or body fluids of organisms, causing various diseases or conditions. It is therefore supposed that inhibition of Aβ formation, accumulation or aggregation can be used as an approach for effectively preventing or treating Aβ-associated diseases or conditions.

The term “prevent” used herein means avoiding or delaying occurrence of a disease or condition in organisms. The term “treat” used herein means slowing or stopping progress of a disease or condition, or making an individual return back to his improved or normal status.

The term “amyloid β peptide (Aβ)-associated diseases or conditions” generally refers to those diseases or conditions that occur relating to formation, accumulation or aggregation of Aβ, and particularly refers to the diseases or conditions that are caused by Aβ. When abnormal formation, accumulation or aggregation is found in a certain proportion of individuals with certain diseases or conditions, the diseases or conditions can be considered as being associated with Aβ. In addition, when Aβ aggregates somewhere that is close to occurrence of pathological features affected in certain diseases or conditions, the diseases or conditions can be also considered as being associated with Aβ.

In the following examples test samples listed in Table 1 were used for carrying out the Aβ experiments, which were compared to a Vehicle control group which was not added with any test samples.

TABLE 1
Test samples

| Symbol | Test sample | Concentration | Source |
|---|---|---|---|
| A | Acteoside | 50 μg/ml | Sinphar Lab., purity 97% |
| I | Isoacteoside | 50 μg/ml | Sinphar Lab., purity 97% |
| A:I | Acteoside + | 40 μg/ml + | Sinphar Lab., purity 97% |
| 40:10 | Isoacteoside | 10 μg/ml | |
| A:I | Acteoside + | 30 μg/ml + | Sinphar Lab., purity 97% |
| 30:20 | Isoacteoside | 20 μg/ml | |
| A:I | Acteoside + | 20 μg/ml + | Sinphar Lab., purity 97% |
| 20:30 | Isoacteoside | 30 μg/ml | |
| A:I | Acteoside + | 10 μg/ml + | Sinphar Lab., purity 97% |
| 10:40 | Isoacteoside | 40 μg/ml | |

### Example 1

### Neuroblastoma Cell Culture

Wild-type human neuroblastoma cells (SH-SY5Y) were cultured in Eagle's Minimum essential Medium (EMEM)/Ham's F12 medium (1:1 mixture) (containing 10% FBS, 10 units/ml penicillin, 10 μg/ml Streptomycin). Wild-type mouse neuroblastoma Neuro-2a cells were cultured in minimum essential medium (MEM) (containing 10% FBS, 10 units/ml penicillin, 10 μg/ml Streptomycin).

### Example 2

### The Effect of Each Test Sample on Extracellular Aβ1-40 Accumulation

The medium of the wild-type human neuroblastoma SH-SY5Y cells in Example 1 were switched into chemical defined medium (EMEM/F12 medium (Cat. No. 12500-062), Hepes 5 mM, Glucose 0.6%, NaHCO₃ 3 mM, Glutamine 2.5 mM, Insulin 25 μg/ml, Transferin 100 μg/ml, Progestrone 20 nM, Putrescine 60 μM, Sodium selenite 30 nM, Heparin 2 μg/ml). Each well contained 1×10⁵ SH-SY5Y cells in 300 μl of culture medium. Thirty minutes later, each well was treated with the test samples given in Table 1 respectively at a total concentration of 50 μg/ml for 24 hours. After that, the level of Aβ1-40 in the medium of each well was analyzed by Human Aβ1-40 immunoassay kits (Catalog #KHB3482 Invitrogen).

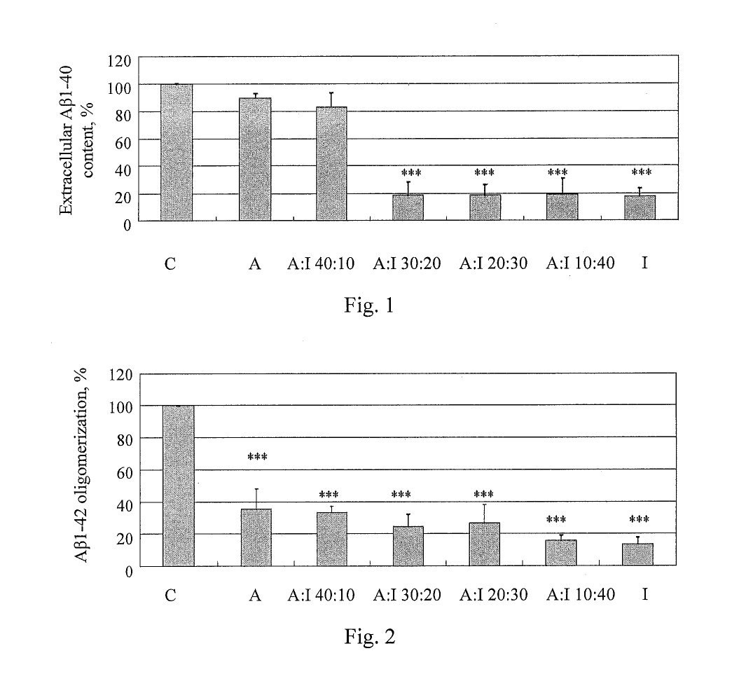

Human neuroblastoma SH-SY5Y cells cause extracellular accumulation of Aβ. FIG. 1 shows the percentage of Aβ1-40 in the medium of each SH-SY5Y well treated with the test samples, based on the amount of Aβ1-40 in the medium of the Vehicle control group (C) which was not treated with any test sample. The results were shown in mean ± standard deviation (SD) form. Significant difference between the Vehicle control group and the test sample-treated groups were indicated by * * * , P<0.001.

Referring to FIG. 1, the test sample A (acteoside) reduces the level of Aβ1-40 in the medium by about 10%, the test sample A:I 40:10 (acteoside 40 μg/ml+isoacteoside 10 μg/ml) reduces the level of Aβ1-40 in the medium by about 22%, the remaining test samples reduce the level of Aβ1-40 in the medium by about 80%. The results in FIG. 1 indicate that the test samples of acteoside+isoacteoside=30 μg/ml+20 μg/ml; 20 μg/ml+30 μg/ml; and 10 μg/ml+40 μg/ml, and the test sample of isoacteoside=50 μg/ml possess significant activity on reducing extracellular Aβ1-40 accumulation.

### Example 3

### The Effect of Each Test Samples on Aβ1-42 Oligomerization

Dried Human Aβ1-42 was taken out from the refrigerator and equilibrated to room temperature. Aβ1-42 was dissolved in 1,1,1,3,3,3-Hexa-fluro-2-propanol (HFIP) to a concentration of 1 mM, and was then placed at room temperature for one hour. The Aβ1-42/HFIP solution was aliquoted by Hamilton syringe, and was then dried under a stream of nitrogen gas, followed by storing at a temperature of −20° C. Aβ1-42 treated with HFIP was dissolved in PBS, and was vibration-incubated with treatment of each test sample at a concentration of 50 μg/ml and at 4° C. for 24 hours to prepare Aβ1-42 oligomers. The level of Aβ1-42 oligomerization was analyzed by thioflavin T fluorescence (Ex=450 nm, Em=482 nm).

FIG. 2 shows the effects of the test samples in Table 1 on Aβ1-40 oligomerization, and the results are shown in percentage based on the control group (C) which was not treated with any test sample. The results in FIG. 2 indicate that all the test samples were found to possess activities on inhibiting Aβ1-42 oligomerization, wherein the test samples of acteoside+isoacteoside=30 μg/ml+20 μg/ml; 20 μg/ml+30 μg/ml; and 10 μg/ml+40 μg/ml, and the test sample of isoacteoside=50 μg/ml possess better activity on inhibiting Aβ1-42 oligomerization. That is to say, these pharmaceutical compositions can be used to prevent or treat Aβ-associated diseases or conditions.

The described Aβ-associated diseases or conditions comprise but not limit to Alzheimer's disease, mild cognitive impairment, Lewy body dementia, Down syndrome, hereditary cerebral hemorrhage with amyloid (HCHWA) Dutch, Parkinsonism-dementia complex on Guam, Cerebral amyloid angiopathy, inclusion body myositis, frontotemporal dementia, age-related macular degeneration, Pick's disease, and others. In addition, even though the described Aβ is exemplified by Aβ1-40 at most or highly fibrillogenic Aβ1-42, the Aβ can also comprise other peptide fragments.

The results in FIGS. 1 and 2 show that isoacteoside possesses a better activity; however, in realizing the application of isoacteoside which is a saccharide-containing molecule is difficult to be chemically synthesized. It is also very costive for obtaining a high purity isoacteoside from the source of a plant. Taking the practical application aspects into consideration such as the economic benefit and the medical treatment effectiveness, the results in FIGS. 1 and 2 indicate that the mixtures of acteoside and isoacteoside possessing an activity comparable to the pure isoacteoside as an amyloid β peptide inhibitor can be used as an alternative of the purified isoacteoside in preventing or treating amyloid β peptide-associated diseases or conditions.

In the following example, the process for preparing a phenylethanoid glycoside-containing preparation disclosed in U.S. Pat. No. 7,087,252 was adopted, which comprises the following steps: a) extracting subterranean portions of (sufleshy stems) of Cistanche tubulosa (Schenk.) Wight with a first polar solvent; b) introducing the resulting extract from step a) into a column which is packed with hydrophobic macro-porous polymeric beads, thereby enabling phenylethanoid glycosides to be adsorbed on the polymeric beads; c) eluting the column by use of a second polar solvent serving as a mobile phase, so that relatively less strongly adsorbed compounds are eluted from the column with most of phenylethanoid glycosides still being adsorbed on the polymeric beads; and d) eluting the column by use of a third polar solvent so as to obtain an eluate which contains phenylethanoid glycosides, wherein the third polar solvent is lower in polarity than the second polar solvent.

The first polar solvent in step a) can be for example water or a mixed solvent of water and ethanol. The second polar solvent in step c) is water. The third polar solvent in step d) can be for example methanol, ethanol, a mixed solvent of water and methanol, or a mixed solvent of water and ethanol, wherein the third polar solvent is a mixed solvent of water and ethanol.

The present invention provides a further purification process to obtain a pharmaceutical composition comprises acteoside and isoacteoside which are the only phenylethanoid glycosides contained therein by directly purifying the aforesaid phenylethanoid glycosides-containing preparation from Cistanche tubulosa (Schenk.) Wight. The further purification process comprises the steps of: e) purifying the aforesaid preparation from Cistanche tubulosa (Schenk.) Wight containing various phenylethanoid glycosides with a macro-porous resin; and f) eluting the macro-porous resin with a fourth polar solvent and a fifth polar solvent in sequence, wherein the fifth polar solvent is lower in polarity than the fourth polar solvent, so that an eluate resulting from the fifth polar solvent elution contains substantially only acteoside and isoacteoside of the phenylethanoid glycosides. In one of the preferred embodiments of the present invention the fourth polar solvent can be for example 25-35% ethanol aqueous solution and the fifth polar solvent can be for example 35-45% ethanol aqueous solution.

Preferably, the hydrophobic macro-porous polymeric beads are cross-linked polyaromatics, and more preferably cross-linked polystyrene or cross-linked copolymer of styrene and divinyl benzene, such as D-101 type or AB-8 type materials.

A pharmaceutical composition contains substantially only acteoside and isoacteoside of the phenylethanoid glycosides can be directly obtained by concentrating or drying the eluate resulting from the fifth polar solvent elution.

### Example 4

### A Pharmaceutical Composition Contains Substantially Only Acteoside and Isoacteoside of the Phenylethanoid Glycosides

10 kg of the flakes of fleshy stems of Cistanche tubulosa (Schenk.) Wight was soaked in water in an amount which was 8 times of the flakes. The flakes was soaked in the water for one hour before being decocted with the water for two hours. The decocted mixture was filtered to obtain a first filtrate. The residue was then decocted with the water in an amount which was 6 times of the residue and the decocted mixture was filtered to obtain a second filtrate. A third filtrate was also obtained by the same procedures as the second filtrate. The three filtrates were combined and concentrated in vacuo to have a specific gravity of 1.10 (50° C.). The filtrate in the concentrated form was mixed with ethanol to form a mixture containing 60% of the ethanol, which was then refrigerated for 12 hours. Thereafter, a supernatant was harvested from the cooled mixture while the residue was filtered to obtain a filtrate, which was combined with the supernatant followed by concentrating in vacuo to obtain an end extract having a specific gravity of 1.10 (50° C.).

6 kg of the end extract was dissolved in water with heating, which was in the same amount of the end extract. The extract solution was then applied into an adsorption column packed with macro-porous adsorption resin. The column was first eluted with water to yield a water eluate in the amount of four times of the fleshy stems, and was than eluted with 40% ethanol to yield a first 40% ethanol eluate in the amount of five times of the fleshy stems. The water eluate was subjected to another round of the adsorption-desorption operations by eluting the column with water in the amount of three times of the fleshy stems and with 40% ethanol in sequence to obtain a second ethanol eluate in the amount of four times of the fleshy stems. The two 40% ethanol eluates were combined, concentrated, and dried to yield a preparation containing phenylethanoid glycosides and having a weight of 1107 g.

A high performance liquid chromatography (HPLC) was carried out under the following conditions: solvent A: acetonitrile containing 0.1% formic acid (CAN); solvent B: MQ-H₂O containing 0.1% formic acid; column: Agilent Zorbax SB-C18 column of 2.1×150 mm, 5 μm; flow rate: 0.3 ml/min; and UV wavelength of 333 nm. The contents of echinacoside, acteoside and isoacteoside of the preparation containing phenylethanoid glycosides were measured, which were calculated as 33.6 wt %, 3.65 wt % and 6.05 wt %, respectively.

200 g of the preparation containing phenylethanoid glycosides was dissolved in 800 g of water, and the resulting solution was introduced into a macro-porous resin to undergo purification, which was eluted with 30% ethanol aqueous solution and 40% ethanol aqueous solution in sequence. A thin layer chromatography was conducted with UV 365 nm to analyze each eluate, wherein the eluate collected from the 30% ethanol aqueous solution does not contain acteoside and isoacteoside, and the eluate collected from the 40% ethanol aqueous solution contain only acteoside and isoacteoside of phenylethanoid glycosides of 23.6 g. In this example, the acteoside in the eluate is 22.5 wt %, and the isoacteoside in the eluate is 36.4 wt %.

Although the present invention has been disclosed by several preferred embodiments described above, they are not for limiting the present invention. Various equivalent replacements and modifications made without departing from the spirit of the present invention by those skilled in the art should be still within the scope of the appended claims.

1-15. (canceled)

16. A method for treating a disease or condition associated with amyloid β peptides in an individual in need thereof, comprising administering to the individual a pharmaceutical composition comprising phenylethanoid glycosides at an amount effective for inhibiting formation, accumulation or aggregation of amyloid β peptides in the individual, wherein the pharmaceutical composition comprises acteoside and isoacteoside as the only phenylethanoid glycosides therein, wherein a weight ratio of the isoacteoside to the acteoside is about 4:1 to about 1:4.

17. The method of claim 16, wherein the disease or condition is related to formation, accumulation or aggregation of the amyloid β peptides.

18. The method of claim 17, wherein the disease or condition is related to extracellular formation, accumulation or aggregation of the amyloid β peptides.

19. The method of claim 16, wherein the amyloid β peptides are Aβ1-40 or Aβ1-42.

20. The method of claim 16, wherein the disease or condition is Alzheimer's disease, mild cognitive impairment, Lewy body dementia, Down syndrome, Hereditary cerebral hemorrhage with amyloid (HCHWA) Dutch, Parkinsonism-dementia complex on Guam, Cerebral amyloid angiopathy, inclusion body myositis, frontotemporal dementia, age-related macular degeneration, or Pick's disease.

21. The method of claim 20, wherein the disease or condition is Alzheimer's disease.

22. The method of claim 16, wherein the pharmaceutical composition is administered to said individual for inhibiting neuronal damage or apoptosis caused by the amyloid β peptides, so as to retain, improve or restore learning and memory abilities of said individual.

23. The method of claim 16, wherein the pharmaceutical composition is administered to said individual in a dosage equivalent to 0.2 mg-4.0 mg of the phenylethanoid glycosides per kg of body weight per day.

24. A method for inhibiting formation, accumulation or aggregation of amyloid β peptides in an individual in need thereof, comprising administering an effective amount of pharmaceutical composition comprising phenylethanoid glycosides to the individual to inhibit the formation, accumulation or aggregation of the amyloid β peptides, wherein the pharmaceutical composition comprises acteoside and isoacteoside as the only phenylethanoid glycosides therein, wherein a weight ratio of the isoacteoside to the acteoside is about 4:1 to about 1:4.

25. The method of claim 24, wherein the pharmaceutical composition is administered to inhibit extracellular formation, accumulation or aggregation of the amyloid β peptides.

26. The method of claim 24, wherein the amyloid β peptides are Aβ1-40 or Aβ1-42.

27. The method of claim 24, wherein the pharmaceutical composition is administered as an additive in food, drinks, chewing gums, patches or skin care products.

28. The method of claim 16, wherein the pharmaceutical composition is the sole active ingredient administered to the individual.

29. The method of claim 16, wherein the pharmaceutical composition comprises a phenylethanoid glycoside preparation extracting from a plant as a source of the isoacteoside to the acteoside, wherein a content of the isoacteoside in the preparation is greater than that of the acteoside.

30. The method of claim 29, wherein the preparation comprises 12-32% of acteoside and 26-46% of the isoacteoside, based on the weight of the preparation.

31. The method of claim 29, wherein the plant is Cistanche tubulosa (Schenk.) Wight.

32. The method of claim 29, wherein the preparation is provided by a process comprising the following steps:
a) extracting fleshy stems of Cistanche tubulosa (Schenk.) Wight with a first polar solvent;
b) introducing the resulting extract from step a) into a column which is packed with hydrophobic macro-porous polymeric beads, thereby enabling phenylethanoid glycosides to be adsorbed on the polymeric beads;
c) eluting the column by use of a second polar solvent serving as a mobile phase, so that relatively less strongly adsorbed compounds are eluted from the column with most of phenylethanoid glycosides still being adsorbed on the polymeric beads; and
d) eluting the column by use of a third polar solvent so as to obtain an eluate which contains phenylethanoid glycosides, wherein the first polar solvent is water, methanol, ethanol, a mixture of water and methanol, or a mixture of water and ethanol; the second polar solvent is water; and the third polar solvent is methanol, ethanol, a mixture of water and methanol, or a mixture of water and ethanol, and the third polar solvent is lower in polarity than the second polar solvent;
e) concentrating the eluate which contains phenylethanoid glycosides, dissolving the concentrate in water, and contacting the aqueous solution with a macro-porous resin, so that the phenylethanoid glycosides are adsorbed on the macro-porous resin; and
f) eluting the macro-porous resin with a fourth polar solvent and a fifth polar solvent in sequence, wherein the fifth polar solvent is lower in polarity than the fourth polar solvent, so that an eluate resulting from the fourth polar solvent elution does not contain acteoside and isoacteoside, and an eluate resulting from the fifth polar solvent elution contain only acteoside and isoacteoside, wherein the fourth polar solvent and the fifth polar solvent are a mixture of water and methanol or a mixture of water and ethanol.

33. The method of claim 32, wherein the fourth polar solvent is 25-35% ethanol aqueous solution and the fifth polar solvent is 35-45% ethanol aqueous solution.

34. The method of claim 24, wherein the pharmaceutical composition is the sole active ingredient administered to the individual.

35. The method of claim 24, wherein the pharmaceutical composition comprises a phenylethanoid glycoside preparation extracting from a plant as a source of the isoacteoside to the acteoside, wherein a content of the isoacteoside in the preparation is greater than that of the acteoside.

36. The method of claim 35, wherein the preparation comprises 12-32% of acteoside and 26-46% of the isoacteoside, based on the weight of the preparation.

37. The method of claim 35, wherein the plant is Cistanche tubulosa (Schenk.) Wight.

38. The method of claim 35, wherein the preparation is provided by a process comprising the following steps:
g) extracting fleshy stems of Cistanche tubulosa (Schenk.) Wight with a first polar solvent;
h) introducing the resulting extract from step a) into a column which is packed with hydrophobic macro-porous polymeric beads, thereby enabling phenylethanoid glycosides to be adsorbed on the polymeric beads;
i) eluting the column by use of a second polar solvent serving as a mobile phase, so that relatively less strongly adsorbed compounds are eluted from the column with most of phenylethanoid glycosides still being adsorbed on the polymeric beads; and
j) eluting the column by use of a third polar solvent so as to obtain an eluate which contains phenylethanoid glycosides, wherein the first polar solvent is water, methanol, ethanol, a mixture of water and methanol, or a mixture of water and ethanol; the second polar solvent is water; and the third polar solvent is methanol, ethanol, a mixture of water and methanol, or a mixture of water and ethanol, and the third polar solvent is lower in polarity than the second polar solvent;
k) concentrating the eluate which contains phenylethanoid glycosides, dissolving the concentrate in water, and contacting the aqueous solution with a macro-porous resin, so that the phenylethanoid glycosides are adsorbed on the macro-porous resin; and
l) eluting the macro-porous resin with a fourth polar solvent and a fifth polar solvent in sequence, wherein the fifth polar solvent is lower in polarity than the fourth polar solvent, so that an eluate resulting from the fourth polar solvent elution does not contain acteoside and isoacteoside, and an eluate resulting from the fifth polar solvent elution contain only acteoside and isoacteoside, wherein the fourth polar solvent and the fifth polar solvent are a mixture of water and methanol or a mixture of water and ethanol.

39. The method of claim 38, wherein the fourth polar solvent is 25-35% ethanol aqueous solution and the fifth polar solvent is 35-45% ethanol aqueous solution.
